(19) United States
(12) Patent Application Publication
(10) Pub. No.: US 2017/0080219 A1
(43) Pub. Date: Mar. 23, 2017

(54) APPARATUS, METHOD AND KIT FOR MANAGEMENT OF A WOUND

(71) Applicant: Anthony J. BALSAMO, Wilkes-Barre, PA (US)

(72) Inventor: Anthony J. BALSAMO, Wilkes-Barre, PA (US)

(21) Appl. No.: 15/312,261

(22) PCT Filed: Jul. 31, 2015
(86) PCT No.: PCT/US15/43094

§ 371 Date: Nov. 18, 2016

Related U.S. Application Data
(60) Provisional application No. 62/031,400, filed on Jul. 31, 2014.

(51) Int. Cl. A61N 1/30; A61M 1/00; A61N 1/04; A61M 3/02; A61M 35/00

(52) U.S. Cl. CPC A61N 1/303; A61M 3/02; A61M 35/00; A61N 1/0448; A61M 1/0058; A61M 3/0204

(57) ABSTRACT

An apparatus, method and kit are provided for managing a wound in an animal subject. More specifically, an expandable device, which is wound conforming, in direct contact with the wound, and drives wound healing ions into wound tissue, a method for driving wound healing ions into the wound tissue by applying an ionizing current, and a kit for driving wound healing ions into the wound tissue powered by a portable power source are provided. The apparatus, method and kit can be used to increase the penetration of a wound healing agent in the wound tissue, and thus potentially decrease healing time, likelihood of infection, and prevalence of chronic wounds.

### CROSS-REFERENCE TO RELATED APPLICATIONS

This application claims priority from U.S. Provisional Patent Application No. 62/031,400, filed Jul. 31, 2014, the disclosure of which is hereby incorporated by reference herein in its entirety.

### TECHNICAL FIELD

The invention relates to an apparatus and method for managing a wound in an animal subject, and a self-contained kit for managing a wound. More specifically, the invention relates to an expandable, electrically conductive wound conforming device, a method for delivering an ionizable wound healing agent into the wound tissue, and a kit comprising a wound conforming device and employing the method for delivering an ionizable wound healing agent for wound management in the field.

### BACKGROUND OF THE INVENTION

Wounds or skin lesions are common injuries that often require immediate medical attention. Once a wound has been cleaned and the bleeding, if any, has been sufficiently slowed, the wound may require a wound healing agent to prevent infection of the affected area. Common wound healing agents include topical antibiotics and nontoxic antiseptics. Even distribution of the wound healing agent over the entire wound tissue can have a positive effect on the rate of healing of a wound.

Despite the use of topical wound healing agents, wound infections and chronic wounds are prevalent in the medical field. Wound infections and chronic wounds can be the result of insufficient treatment of an open wound. Commonly used topical antibiotics and nontoxic antiseptics may be at a disadvantage due to the wound tissue's limitation in absorbing the topical wound healing agent. Deep tissue absorption of a wound healing agent can potentially decrease the likelihood of an infection and the prevalence of chronic wounds by treating the wound beneath the surface. In addition, healing time of a wound may be decreased by eliminating the need for further treatments.

Serious wounds require immediate medical attention that is not always readily available. For example, wounds are common in the military field where hospitals and intricate medical equipment may be inaccessible. A topical wound healing agent applied in this environment may not be sufficient to prevent the development of a life-threatening wound infection or chronic wound. By delivering a wound healing agent deep below the surface of the wound tissue, the serious wound may be sufficiently managed until a more thorough medical procedure can be performed.

### SUMMARY OF THE INVENTION

One aspect of the invention comprises an apparatus for managing a wound in an animal subject. The apparatus generally comprises a source of a wound healing agent, an irrigation system to deliver the wound healing agent into the wound cavity, an expandable wound conforming device to overlie and contact the wound tissue cavity, and a power source in electrical communication with the expandable wound conforming device to deliver a current to the wound healing agent.

Another aspect of the invention comprises a method for managing a wound in an animal subject. The method generally comprises delivering a wound healing agent into the wound cavity, wherein the wound healing agent contains an ionizable compound, placing an expandable wound conforming device over the wound tissue, expanding the expandable wound conforming device to overlie and contact the wound tissue, and applying a current to the expandable wound conforming device. According to one embodiment of the invention, the method for managing a wound in an animal subject can further comprise shrinking the expandable wound conforming device and removing the excess wound healing agent and debris. Optionally, some or all of these steps can be repeated until the wound is sufficiently healed.

Another aspect of the invention comprises a self-contained kit for managing a wound in an animal subject. The kit generally comprises a reservoir containing a wound healing agent, wherein the wound healing agent contains an ionizable compound, an injector for delivering the wound healing agent from the reservoir to the wound cavity, an expandable wound conforming device to overlie and contact the walls of the wound tissue cavity, and a self-contained, portable power source in electrical communication with the expandable wound conforming device.

### BRIEF DESCRIPTION OF THE DRAWINGS

The foregoing summary, as well as the following detailed description of the invention, will be better understood when read in conjunction with the accompanying drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a schematic illustration of an apparatus for managing a wound in an animal subject, according to one embodiment of the invention, wherein the expandable wound conforming device is in the shrunken configuration.

FIG. 2 is a schematic illustration of the apparatus of FIG. 1, wherein the expandable wound conforming device is in the expanded configuration.

FIG. 3A is a schematic illustration of a first step of a method for managing a wound in an animal subject, according to one embodiment of the invention, wherein wound healing agent is delivered to the wound cavity.

FIG. 3B is a schematic illustration of a second step of a method for managing a wound in an animal subject, wherein an expandable wound conforming device is placed over the wound cavity.

FIG. 3C is a schematic illustration of a third step of a method for managing a wound in an animal subject, the expandable wound conforming device is expanded to overlie and contact the wound tissue and trap the wound healing agent between the expandable wound conforming device and the wound tissue.

FIG. 3D is a schematic illustration of a fourth step of the method for managing a wound in an animal subject, wherein a current is applied to the expandable wound conforming device to deliver a current to the wound healing agent to drive wound healing ions into the wound tissue.

FIG. 4A is a schematic illustration of a further step of a method for managing a wound in an animal subject, wherein the wound conforming device is shrunk.

FIG. 4B is a schematic illustration of the last step of a method for managing a wound in an animal subject, wherein the excess wound healing agent and wound debris are removed from the wound cavity.

FIG. 5 is a schematic illustration of a kit for managing a wound in an animal subject, according to one embodiment of the invention, wherein the expandable wound conforming device is in the shrunken configuration.

FIG. 6 is a schematic illustration of the kit of FIG. 5, wherein the expandable wound conforming device is in the expanded configuration.

FIG. 7A is an image of the penetration of SDC in an agarose gel after 15 minutes without applying an electrophoretic current.

FIG. 7B is an image of the silver ions in the agarose gel of FIG. 7A.

FIG. 8A is an image of the penetration of SDC in an agarose gel after applying an electrophoretic current at 10 V for 15 minutes.

FIG. 8B is an image of the silver ions in the agarose gel of FIG. 8A.

FIG. 9 is an image of the penetration of SDC in a sample of raw pork after 10 minutes without applying an iontophoretic current.

FIG. 10 is an image of the penetration of SDC in a sample of raw pork after applying a 125 μA iontophoretic current at 10 V for 10 minutes.

### DETAILED DESCRIPTION OF THE INVENTION

The invention relates to an apparatus and method for managing a wound in an animal subject, and a self-contained kit for managing a wound. More specifically, the invention relates to an expandable, electrically conductive wound conforming device, a method for delivering an ionizable wound healing agent into the wound tissue, and a kit comprising a wound conforming device and employing the method for delivering an ionizable wound healing agent for wound management in the field. The invention can be used to increase the penetration of a wound healing agent into the wound tissue, and thus potentially decrease healing time, likelihood of infection, and prevalence of chronic wounds. The invention can also be used to degrade biofilm tissue in and around a wound.

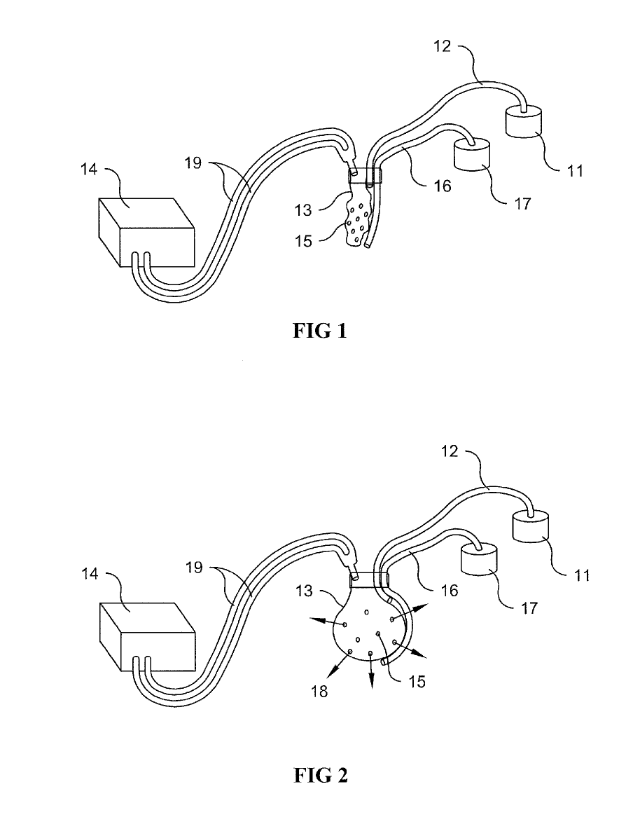

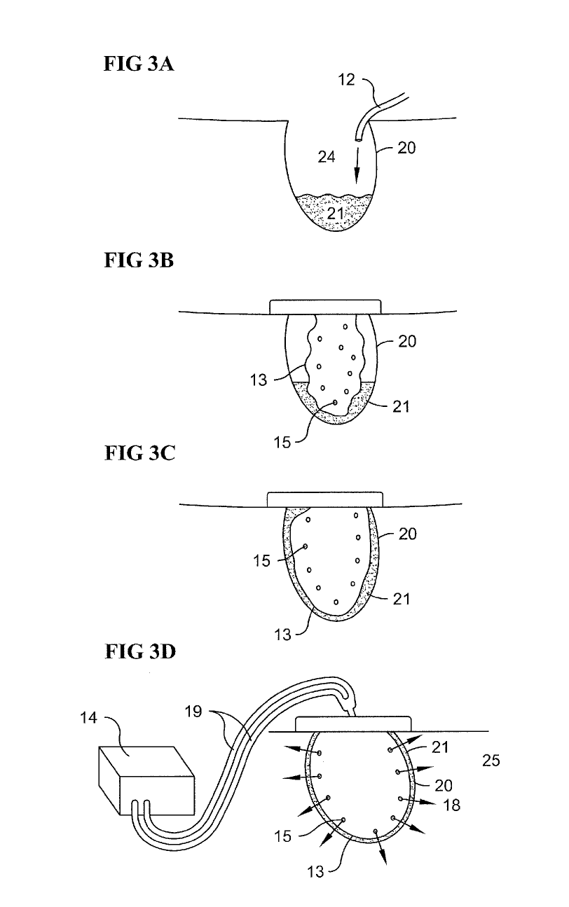

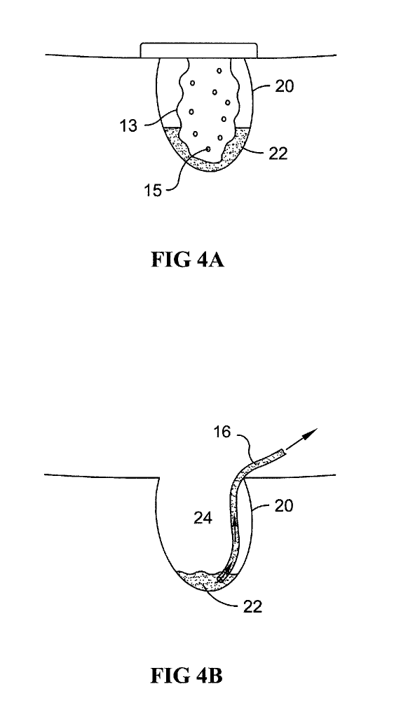

One aspect of the invention comprises an apparatus for managing a wound in an animal subject. As used herein, an “animal subject” is any animal or human in need of wound management. As representative of one aspect of the invention, FIGS. 1-2 illustrate an embodiment of the apparatus for managing a wound in an animal subject, for example as in the method illustrated in FIGS. 3A-3D and 4A-4B. The apparatus generally comprises a source of a wound healing agent 11, an irrigation system to deliver the wound healing agent 21 into the wound cavity 24, an expandable wound conforming device 13 to overlie and contact the wound tissue 20, and a power source 14 in electrical communication 19 with the expandable wound conforming device 13.

The source of the wound healing agent 11 may be a reservoir, such as a flask, a bottle or a vial, wherein the wound healing agent 21 is delivered to the wound cavity 24 by a wound healing agent delivery line 12, as illustrated in FIG. 1. Equally, the source of wound healing agent 11 may be an injector either connected by a wound healing agent delivery line 12 or adjacent to the expandable wound conforming device 13, and therefore adjacent to the wound cavity 24.

The wound healing agent 21 can be liquid or semi-liquid, including gels, or any sufficiently fluid form that may be delivered by an irrigation system and distributed to contact a substantial portion of the wound tissue by expanding the expandable wound conforming device. The wound healing agent 21 can be an aqueous solution, can be prepared from an anhydrous powder and water, or can be prepared by any other means of preparing a liquid or semi-liquid wound healing agent. According to one embodiment of the invention, the wound healing agent 21 comprises a compound having metal cations or anions. For example, the metal cations may be selected from Ag, Cu, Au and Pt. Equally, the wound healing agent 21 may comprise a compound having anions, for example I. According to a preferred embodiment of the invention, the wound healing agent 21 is an antimicrobial ion solution, for example silver dihydrogen citrate (SDC). It will be understood that the wound healing agent 21 can be any wound healing agent that would be employed to advance the healing of a wound and contains an ionizable compound or can be carried as a charged particle.

As illustrated in FIG. 2, the expandable wound conforming device 13 can be expanded to conform to the particular shape of the wound cavity 24. Upon doing so, the expandable wound conforming device 13 may trap the wound healing agent 21 between the expandable wound conforming device 13 and the wound tissue 20. The expansion of the expandable wound conforming device 13, if necessary, will ensure the wound healing agent 21 is delivered to all the exposed wound tissue 20 of the wound cavity 24, and thus maximize the contact of the wound tissue 20 with the wound healing agent 21. By maximizing the contact of the wound tissue 20 with the wound healing agent 21, the effectiveness of the wound healing agent 21 can potentially be increased, due to increased penetration into the tissue. When expanded, as illustrated in FIG. 2, the expandable wound conforming device 13 may have a substantially spherical shape, however it will be understood that the shape and dimensions of the expandable wound conforming device 13 can be varied, and are intended to conform as closely as possible to the shape of the wound cavity. For example, a flatter expandable wound conforming device can be used to treat a superficial wound and requires less expansion, while a more oval-shaped expandable wound conforming device can be used to treat a deeper wound and requires more expansion.

According to one embodiment of the invention, all or a portion of the expandable wound conforming device 13 is an inflatable membrane, preferably a non-porous inflatable membrane. The inflatable membrane may be inflated by any means, for example by devices such as a connected air tank or a hand pump. Equally, the expandable wound conforming device 13 may be mechanically expanded with moving parts that expand to reach the exposed wound tissue 20.

The expandable wound conforming device 13 can further contain at least one embedded electrode 15. The at least one embedded electrode 15 can deliver a current 18 to the wound healing agent 21 to drive wound healing ions 25 of the wound healing agent 21 into the wound tissue 20.

According to another embodiment of the invention, the expandable wound conforming device 13 may be an electrically conductive polymer membrane. The electrically conductive polymer membrane can deliver a current 18 to the wound healing agent 21 to drive wound healing ions 25 of the wound healing agent 21 into the wound tissue 20. For example, all or a portion of the expandable wound conforming device 13 may be a conductive metallic membrane, such as a Mylar® membrane having a metallic coating. The electrically conductive polymer membrane may be inflated, for example by a connected air tank or a hand pump, or may be mechanically expanded.

The current 18 that drives the wound healing ions 25 of the wound healing agent 21 into the wound tissue 20 is delivered by the power source 14 in electrical communication 19 with the expandable wound conforming device 13. A current of 0.5 mA-3.0 mA is sufficient to reach a desired penetration depth of the wound healing agent 21 in a tissue. It will be understood that the power source 14 can be any power source capable of delivering the necessary current and voltage to the expandable wound conforming device 13. More specifically, the power source 14 is capable of delivering a current of 0.5 mA-16 mA to the electrodes 15 in the expandable wound conforming device 13 or to the electrically conductive polymer membrane. The maximum current of 16 mA is safe for human contact, and well below the let-go threshold. Preferably, the power source 14 is capable of delivering 0.5 mA-16 mA of current at 10 V. For example, the power source can be an electrophoresis power supply, such as those distributed by Bio-Rad, an iontophoresis power supply, or a self-contained battery pack, such as the electrophoresis battery pack distributed by Invitrogen.

The irrigation system delivers the wound healing agent 21 from a reservoir containing the wound healing agent 11 to the wound cavity 24. According to one embodiment of the invention, as illustrated in FIGS. 1-2, the irrigation system may be further adapted to remove excess wound healing agent and wound debris by suction. It will be understood that the suction is provided by any means capable of providing suction, for example a vacuum pump or a hand pump. The irrigation system can further be comprised of independent delivery and suction lines 12 and 16, respectively, so as to not contaminate the fresh wound healing agent 21 with the excess wound healing agent and wound debris 22 that may be removed from the wound cavity 24 by the irrigation system.

Another aspect of the invention comprises a method for managing a wound in an animal subject. The method generally comprises delivering a wound healing agent 21 into the wound cavity 20, wherein the wound healing agent 21 contains an ionizable compound, and delivering a current 18 to the wound healing agent 21 to drive wound healing ions 25 of the wound healing agent 21 into the wound tissue 20.

As representative of one embodiment of the invention, FIGS. 3A-3D and 4A-4B illustrate a method for managing a wound in an animal subject. The illustrated embodiment generally comprises delivering a wound healing agent 21 into the wound cavity 24, wherein the wound healing agent 21 contains an ionizable compound, placing an expandable wound conforming device 13 over the wound tissue 20, expanding the expandable wound conforming device 13 to overlie and contact the wound tissue 20, and applying a current 18 to the expandable wound conforming device 13. It will be understood that the order of steps in the following description of a method for managing a wound in an animal subject represents one embodiment of the present invention and may be altered to achieve a desired result.

It will be understood that the wound healing agent 21 can be any wound healing agent that would be employed to advance the healing of a wound and contains an ionizable compound or can he carried as a charged particle. The wound healing agent 21 may be any of the embodiments of a wound healing agent previously described.

One step of the method for managing a wound in an animal subject, as illustrated in FIG. 3A, comprises delivering the wound healing agent 21 to the wound cavity 24. The wound healing agent 21 can be delivered to the wound cavity 24 by any means, for example by an irrigation system designed to deliver the wound healing agent 21, by a syringe, or by manually decanting.

Another step of the method for managing a wound in an animal subject, as illustrated in FIG. 3B, comprises placing the expandable wound conforming device 13 over the wound cavity 24. It will be understood that placement of this step can be either before or after the step of delivering the wound healing agent to the wound. The expandable wound conforming device 13 can be any wound conforming device capable of expanding to reach all the exposed wound tissue 20 of the wound cavity 24. The expandable wound conforming device 13 may be any of the embodiments of an expandable wound conforming device previously described. The expandable wound conforming device 13 may be expanded mechanically or manually.

As illustrated in FIG. 3C, another step of the method for managing a wound in an animal subject comprises expanding the expandable wound conforming device 13 to overlie and contact the wound tissue 20. The expansion of the expandable wound conforming device 13 traps the wound healing agent 21 between the expandable wound conforming device 13 and the wound tissue 20, allowing the wound healing agent 21 to be delivered to all the exposed wound tissue 20 of the wound cavity 24, and thus maximizing the contact of the wound tissue 20 with the wound healing agent 21.

Another step of the method for managing a wound in an animal subject, as illustrated in FIG. 3D, comprises applying a current 18 to the wound conforming device 13 to deliver a current 18 to the wound healing agent 21 to drive wound healing ions 25 of the wound healing agent 21 into the wound tissue 20. The current 18 can be applied by any power source 14 in electrical communication 19 with the expandable wound conforming device 13 and capable of delivering the necessary current and voltage to drive wound healing ions 25 of the wound healing agent 21 into the wound tissue 20. The expandable wound conforming device 13 may contain embedded electrodes 15 that deliver the current 18 to the wound healing agent 21 or may he an electrically conductive polymer membrane capable of evenly distributing the current 18, for example a conductive metallic membrane, such as a Mylar® membrane having a metallic coating.

According to one embodiment of the invention, the method for managing a wound in an animal subject can further comprise shrinking the expandable wound conforming device 13, as illustrated in FIG. 4A, and removing the excess wound healing agent and debris 22, as illustrated in FIG. 4B. The expandable wound conforming device 13 may be shrunk by deflating or mechanically compressing.

Shrinking the expandable wound conforming device 13 may allow the excess wound healing agent and wound debris 22 to pool at the bottom of the wound cavity 24. The excess wound healing agent is any amount of the wound healing agent that has not penetrated the wound tissue 20. The wound debris is any animal tissue or fluid (exudate) that has been debrided, exuded or otherwise dislodged from the wound tissue 20. The excess wound healing agent and wound debris 22 can be removed by any means, including, but not limited to suction, for example provided by a vacuum or a hand pump.

According to yet another embodiment of the invention, the method for managing a wound in an animal subject can further comprise repeating some or all of the steps of delivering the wound healing agent 21 into the wound cavity 24, placing the expandable wound conforming device 13 over the wound cavity 24, expanding the expandable wound conforming device 13 to overlie and contact the wound tissue 20, applying the current 18 to the expandable wound conforming device 13, shrinking the expandable wound conforming device 13 and removing the excess wound healing agent and debris 22. The repetition of some or all of the foregoing steps may take place as often as possible or as necessary until the wound is sufficiently healed. As used herein, the term “sufficiently healed” can refer to any desired level of healing of the wound, or simply sufficient penetration of the wound healing agent 21 into the wound tissue 20, and need not refer to any visibly noticeable healing of the wound tissue 20.

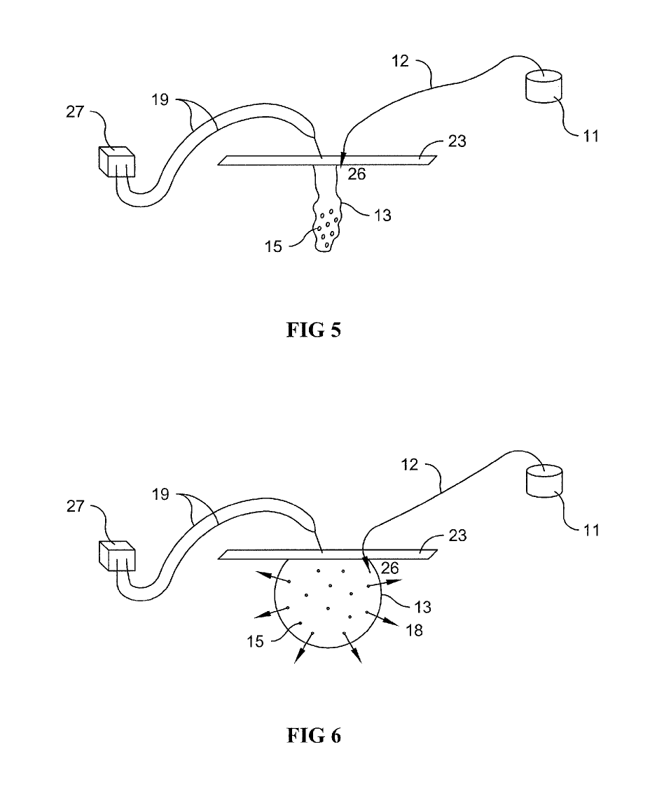

Another aspect of the invention comprises a self-contained kit for managing a wound in an animal subject. As representative of one aspect of the invention, FIGS. 5-6 illustrate an embodiment of the kit for managing a wound in an animal subject. The kit generally comprises a reservoir 11 containing a wound healing agent, wherein the wound healing agent 21 contains an ionizable compound, an injector 26 for delivering the wound healing agent from the reservoir to the wound cavity, an expandable wound conforming device 13 to overlie and contact the wound tissue 20. and a self-contained, portable power source 27 in electrical communication 19 with the expandable wound conforming device 13.

As illustrated in FIG. 5, the injector 26 delivers the wound healing agent 21 from the reservoir 11 containing the wound healing agent to the wound cavity 24. The reservoir 11 may be a flask, a bottle, a vial, a packet, or other receptacle connected by a wound healing agent delivery line 12. The reservoir can be compactly or integrally located in the kit, for example in a bandage or other element. Equally, the reservoir 11 containing the wound healing agent may be a syringe connected directly to the injector 26 or placed adjacently to the expandable wound conforming device 13 for manual injection.

In one embodiment of the invention, the wound healing agent 21 may be substantially similar to the wound healing agent previously described. Equally, the expandable wound conforming device 13 may be substantially similar to those previously described.

The current 18 that drives the wound healing ions 25 of the wound healing agent 21 into the wound tissue 20 is delivered by the self-contained, portable power source 27 in electrical communication 19 with the expandable wound conforming device 13. In a preferred embodiment of the invention, the self-contained, portable power source 27 is a battery pack. For example, the power source can be a self-contained battery pack, such as the electrophoresis battery pack distributed by Invitrogen. It will be understood that the power source 27 can be any self-contained, portable power source capable of delivering the necessary current and voltage to the expandable wound conforming device 13. More specifically, the self-contained, portable power source 27 is capable of delivering a current of 0.5 mA-16 mA to the electrodes 15 in the expandable wound conforming device 13 or to the electrically conductive polymer membrane.

According to one embodiment of the invention, the self-contained kit for managing a wound in an animal subject further comprises an adhesive bandage 23 for adhering the kit to an area around the wound cavity 24. The adhesive bandage 23 allows for simple and rapid management of a wound in the field, until the wound can be further treated in a medical care setting. Equally, the self-contained kit for managing a wound in an animal subject may further comprise other elements capable of adhering the kit to an area around the wound cavity 24, for example gauze pads, rolled gauze or cloth tape.

### EXPERIMENTAL EXAMPLES

Wound Healing Agent Contact with Wound Tissue

In a demonstration of maximizing the contact of the wound healing agent to the wound tissue, an apparatus for managing a wound in an animal subject was placed over a flask, the flask representing the wound cavity. The expandable wound conforming device was placed in the flask and the wound healing agent was delivered to the flask by an irrigation system. The wound healing agent pooled at the bottom of the flask and did not contact the entire inner surface of the flask. The expandable wound conforming device was then expanded by inflation to substantially fill the flask.

Upon expansion of the expandable wound conforming device, the wound healing agent contacted substantially the entire inner surface of the flask, representing the contact that can be made with wound tissue.

The expandable wound conforming device was then shrunk, and the wound healing agent, representing the excess wound healing agent and debris, was removed from the flask by suction.

### Electrophoretic Migration of SDC in an Agarose Gel

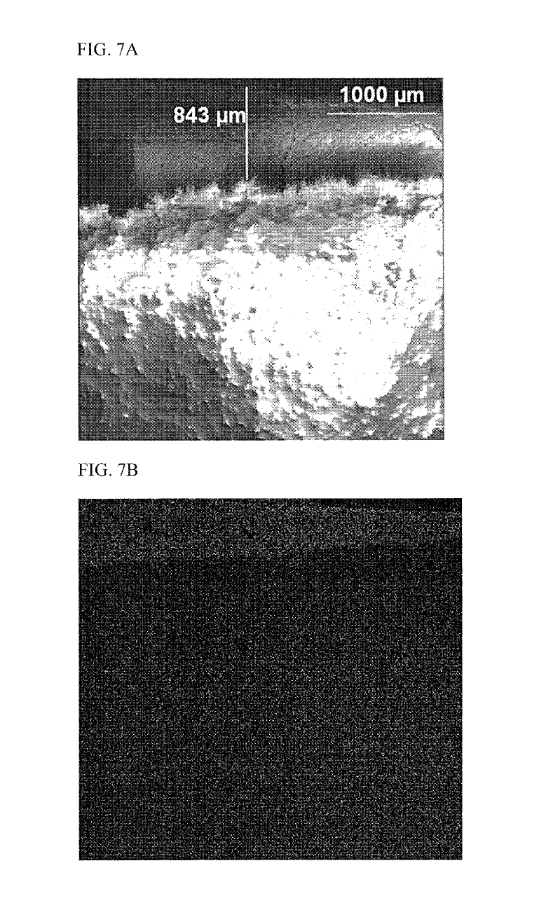

Electrophoretic migration of SDC was tested in an agarose gel. SDC was added to the surface of an agarose gel and allowed to permeate the gel for 15 minutes. FIGS. 7A-7B show the penetration depth of SDC in the agarose gel with no electrophoretic field. The silver ions in FIG. 7A were identified by X-ray microanalysis, and were determined to have reached a depth of 843 μm.

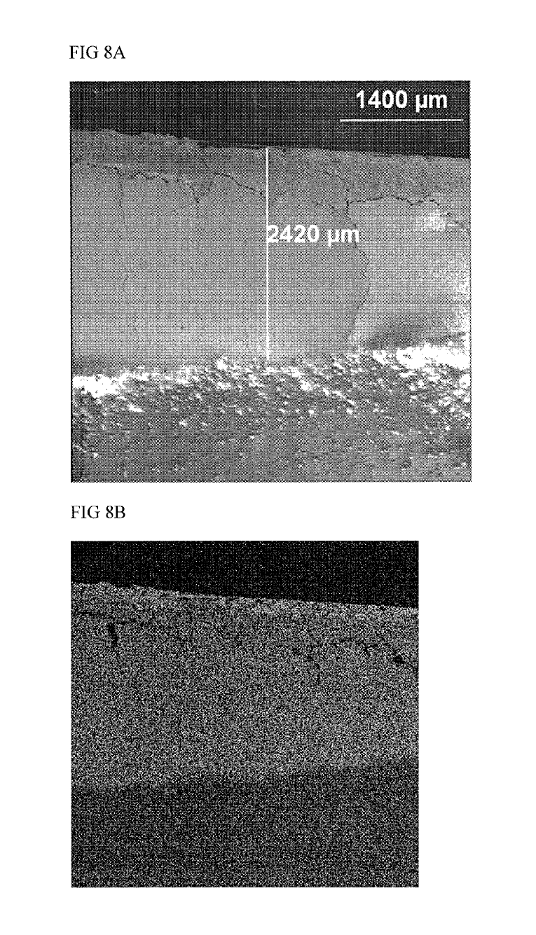

SDC was added to a second agarose gel and an electrophoretic field of 10V was applied to the vertically oriented 8 cm gel for 15 minutes. FIGS. 8A-8B show the penetration depth of SDC in the agarose gel with the 10V electrophoretic field. The silver ions in FIG. 8A were identified by X-ray microanalysis, and were determined to have reached a depth of 2420 μm. By applying the electrophoretic field, SDC was able to penetrate the agarose gel almost 3 times as deeply in the same amount of time, representing the increased ability of a wound healing agent comprising a compound having metal cations, anions or an antimicrobial ion solution to penetrate a wound tissue upon delivering a current.

### Iontophoretic Migration of SDC in a Sample of Raw Pork

Iontophoretic migration of SDC was tested in a 1 cubic centimeter sample of raw pork. A 1 mL sample of SDC was added to the surface of a 1 cubic centimeter sample of raw pork held in a cuvette. In the control sample, the SDC was allowed to permeate the raw pork for 10 minutes with no current applied. In an experimental sample as above, a constant iontophoretic current of 125 μA at 10 V was applied to the sample for 10 minutes. The current/voltage combination was chosen because it was shown in previous experiments that it caused no heating or denaturing of an albumin solution.

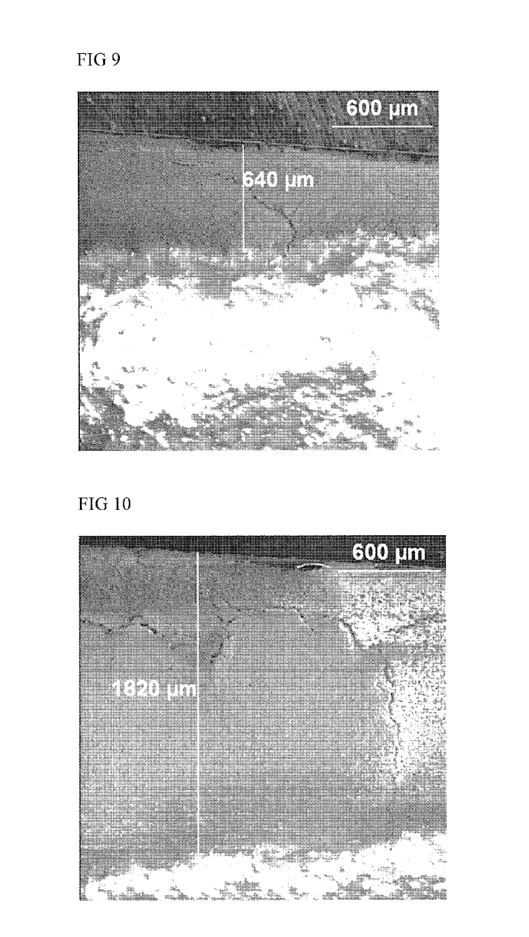

The above-treated samples were removed from the cuvettes and sliced vertically with a scalpel to give cross sections. The cross sections were analyzed in a scanning electron microscope with energy dispersive X-ray detection to produce FIGS. 9 and 10. FIG. 9 shows the penetration depth of SDC in the sample of raw pork with no iontophoretic field is approximately 640 μm. FIG. 10 shows the penetration depth of SDC in the sample of raw pork with the applied iontophoretic field is approximately 1820 μm.

It will be appreciated by those skilled in the art that changes could be made to the embodiments and examples described above without departing from the broad inventive concept thereof. It is understood, therefore, that this invention is not limited to the particular embodiments disclosed, but it is intended to cover modifications within the spirit and scope of the present invention as defined by the appended claims.

1. An apparatus for managing a wound in an animal subject, the apparatus comprising:
a. a reservoir containing a liquid or semi-liquid wound healing agent;
b. an irrigation system to deliver the liquid or semi-liquid wound healing agent into a cavity of the wound, wherein the liquid or semi-liquid wound healing agent contains an ionizable compound;
c. an expandable wound conforming device to contact tissue of the wound cavity and trap the liquid or semi-liquid wound healing agent between the expandable wound conforming device and the wound tissue; and
d. a power source in electrical communication with the expandable wound conforming device to deliver a current to the liquid or semi-liquid wound healing agent to drive wound healing ions of the liquid or semi-liquid wound healing agent into the wound tissue,
wherein the expandable wound conforming device comprises a non-porous inflatable membrane.

2. (canceled)

3. The apparatus of claim 1 wherein the expandable wound conforming device comprises an electrically conductive polymer membrane.

4. The apparatus of claim 1 wherein the expandable wound conforming device contains at least one embedded electrode.

5. The apparatus of claim 1 wherein the irrigation system is further adapted to remove excess liquid or semi-liquid wound healing agent and wound debris by suction.

6. The apparatus of claim 5 wherein the irrigation system is comprised of independent delivery and suction lines.

7. The apparatus of claim 1 wherein the liquid or semi-liquid wound healing agent comprises a compound having metal cations or anions.

8. The apparatus of claim 1 wherein the liquid or semi-liquid wound healing agent is an antimicrobial ion solution.

9. (canceled)

10. A method for managing a wound in an animal subject, the method comprising steps of:
a. delivering a liquid or semi-liquid wound healing agent into a cavity of the wound, wherein the liquid or semi-liquid wound healing agent contains an ionizable compound;
b. placing an expandable wound conforming device over the wound tissue;
c. expanding the expandable wound conforming device to contact tissue of the wound cavity and trap the liquid or semi-liquid wound healing agent between the expandable wound conforming device and the wound tissue; and
d. applying a current to the expandable wound conforming device to deliver a current to the liquid or semi-liquid wound healing agent to drive wound healing ions of the liquid or semi-liquid wound healing agent into the wound tissue,
wherein the expandable wound conforming device comprises a non-porous inflatable membrane.

11. The method of claim 10 further comprising steps of:
e. shrinking the wound conforming device; and
f. removing excess liquid or semi-liquid wound healing agent and wound debris.

12.-13. (canceled)

14. The method of claim 10 wherein the expandable wound conforming device comprises an electrically conductive polymer membrane.

15. The method of claim 10 wherein the expandable wound conforming device contains at least one embedded electrode.

16. (canceled)

17. The method of claim 10 wherein the liquid or semi-liquid wound healing agent is delivered by an irrigation system.

18. The method of claim 10 wherein the liquid or semi-liquid wound healing agent comprises a compound having metal cations or anions.

19. The method of claim 10 wherein the liquid or semi-liquid wound healing agent is an antimicrobial ion solution.

20. (canceled)

21. The method of claim 11 wherein the excess liquid or semi-liquid wound healing agent is removed by suction.

22.-23. (canceled)

24. The method of claim 7 wherein the liquid or semi-liquid wound healing agent contains ions selected from the group consisting of Ag, Cu, Au, Pt, and I.

25. (canceled)

26. The apparatus of claim 8 wherein the antimicrobial ion solution is silver dihydrogen citrate.

27.-37. (canceled)

38. The apparatus of claim 3 wherein the expandable wound conforming device is a mylar membrane having a metallic coating.

39. The method of claim 18 wherein the liquid or semi-liquid wound healing agent contains ions selected from the group consisting of Ag, Cu, Au, Pt, and I.

40. The method of claim 19 wherein the antimicrobial ion solution is silver dihydrogen citrate.

41. The method of claim 13 wherein the expandable wound conforming device is a mylar membrane having a metallic coating.

42. The method of claim 11 wherein the liquid or semi-liquid wound healing agent is delivered by an irrigation system, the excess liquid or semi-liquid wound healing agent is removed by suction, and the irrigation system is comprised of independent delivery and suction lines.
